(19) United States
(12) Patent Application Publication
(10) Pub. No.: US 2009/0112241 A1
(43) Pub. Date: Apr. 30, 2009

(54) Ear cleaning device

(76) Inventors: Israel Bar, Moshav Avigdor (IL); Rali Koren, Moshav Avigdor (IL)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(21) Appl. No.: 11/989,049

(22) PCT Filed: Jul. 18, 2006
(86) PCT No.: PCT/IL2006/000827

§ 371 Date: Nov. 5, 2008

Related U.S. Application Data
(60) Provisional application No. 60/700,543, filed on Jul. 18, 2005.

(51) Int. Cl. A61F 11/00

(52) U.S. Cl. 606/162

(57) ABSTRACT

An ear cleaning device including a body including a finger engagement portion, a connecting neck portion and an ear engagement portion, the body being configured such that finger pressure exerted on the finger engagement portion produces expansion of the ear engagement portion.

### REFERENCE TO RELATED APPLICATIONS

Reference is made to U.S. Provisional Patent Application No. 60/700,543, filed Jul. 18, 2005, entitled EAR CLEANING DEVICE, the disclosure of which is hereby incorporated by reference and priority of which is hereby claimed pursuant to 37 CFR 1.78(a) (4) and (5)(i).

### BRIEF DESCRIPTION OF THE INVENTION

The present invention relates to ear cleaning devices.

### BACKGROUND OF THE INVENTION

The following U.S. Patents and U.S. Patent Publications are believed to represent the current state of the art:

U.S. Pat. Nos.4,718,889; 6,736,826 and 5,107,861.

### SUMMARY OF THE INVENTION

The present invention seeks to provide an improved ear cleaning device.

There is thus provided in accordance with a preferred embodiment of the present invention an ear cleaning device including a body including a finger engagement portion, a connecting neck portion and an ear engagement portion, the body being configured such that finger pressure exerted on the finger engagement portion produces expansion of the ear engagement portion.

In accordance with a preferred embodiment of the present invention the ear engagement portion is provided with at least one ear canal engagement enhancing protrusion on an outer surface thereof. Preferably, the ear engagement portion is provided with brushlike filaments for ear wax engagement.

In accordance with another preferred embodiment of the present invention the ear engagement portion is inflatable by compression of the finger engagement portion. Additionally or alternatively, the finger engagement portion, connecting neck portion and ear engagement portion are integrally formed of a resilient material. Preferably, the connecting portion is less resilient than the finger engagement portion and the ear engagement portion.

In accordance with yet another preferred embodiment of the present invention the finger engagement portion has a generally bulbous configuration. Preferably, the finger engagement portion has a larger interior volume than ear engagement portion.

In accordance with still another preferred embodiment of the present invention the finger engagement portion is formed with at least one finger engagement enhancing protrusion on an outer surface thereof. Preferably, the ear engagement portion is adapted to be inserted into an ear canal when in a first, generally non-expanded configuration and is adapted to be expanded to gently engage the ear canal.

### BRIEF DESCRIPTION OF THE DRAWINGS

The present invention will be understood and appreciated more fully from the following detailed description, taken in conjunction with the drawings in which:

FIGS. 1 & 2 are simplified pictorial and sectional illustrations of an ear cleaning device constructed and operative in accordance with a preferred embodiment of the present invention in a first, non-compressed, operative orientation;

FIGS. 3 & 4 are simplified pictorial and sectional illustrations of the ear cleaning device of FIGS. 1 & 2 in a second, compressed, operative orientation;

FIG. 5 is a simplified pictorial illustration of the ear cleaning device of FIGS. 1-4 in the first, non-compressed, operative orientation about to be inserted into an ear canal; and

FIG. 6 is a simplified pictorial illustration of the ear cleaning device of FIGS. 1-4 in the second, compressed, operative orientation inserted into an ear canal.

### DETAILED DESCRIPTION OF A PREFERRED EMBODIMENT

Reference is now made to FIGS. 1-6, which illustrate an ear cleaning device constructed and operative in accordance with a preferred embodiment of the present invention. As seen particularly clearly in FIGS. 1-4, the ear cleaning device comprises a sealed, flexible and resilient body 10, preferably filled with air, having a finger engagement portion 12, a connecting neck portion 13 and an ear engagement portion 14, preferably integrally formed therewith. Body 10 is preferably formed of latex, plastic or any other suitable resilient material. The connecting neck portion 13 is preferably thicker and somewhat less resilient than the finger engagement portion 12 and the ear engagement portion 14.

The finger engagement portion 12 preferably has a generally bulbous configuration and a larger interior volume than ear engagement portion 14 and may be formed with one or more finger engagement enhancing protrusions 16 on an outer surface thereof.

The ear engagement portion 14, the interior of which communicates with the interior of finger engagement portion 12, is preferably formed with one or more ear canal engagement enhancing protrusions 18 on an outer surface thereof. Protrusions 18 may include or have mounted thereon brushlike filaments 20 or other ear wax engaging elements.

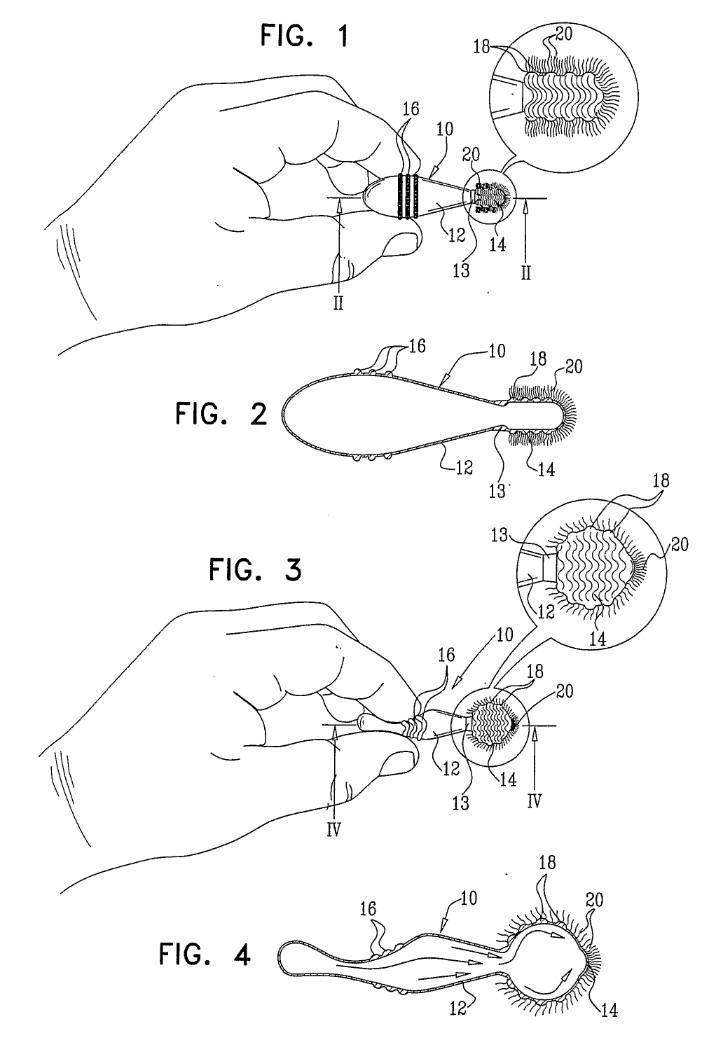

As can be seen from a consideration of FIGS. 1-4, by squeezing on finger engagement portion 12, a user causes expansion of ear engagement portion 14, from a first, generally non-expanded configuration shown in FIGS. 1 & 2 to a second, generally expanded configuration, shown in FIGS. 3 & 4.

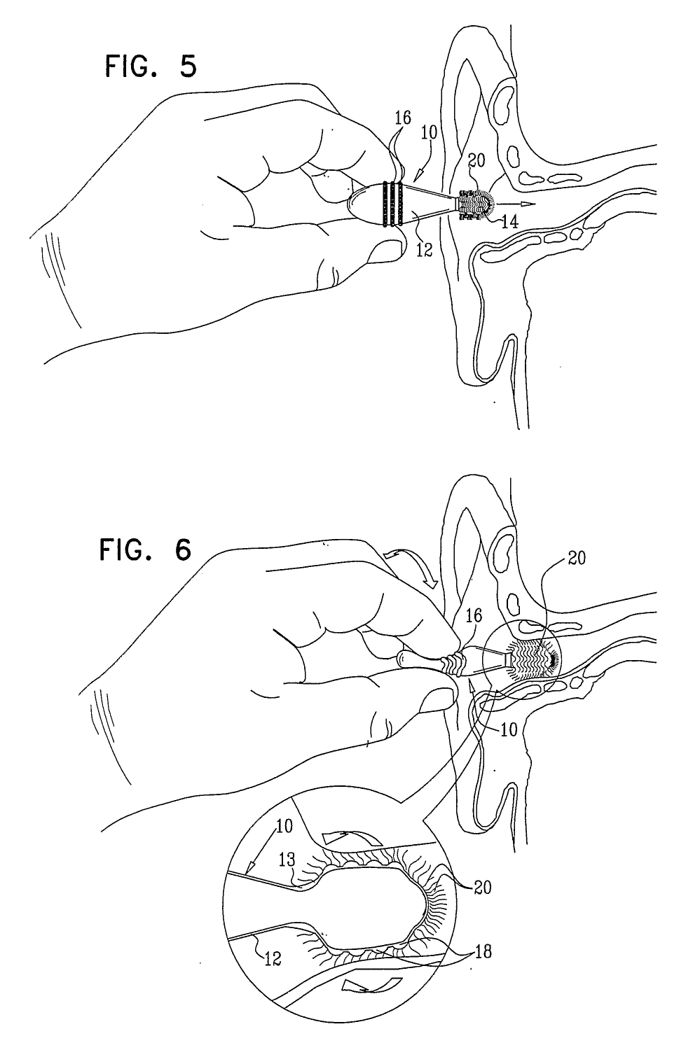

As may be appreciated from a consideration of FIGS. 5 and 6, in use, the ear cleaning device is preferably inserted into an ear canal when in its first, generally non-expanded configuration, as shown in FIG. 5. Once the ear engagement portion 14 is located at a suitable position within the ear canal, the user may squeeze the finger engagement portion 12, causing expansion of the ear engagement portion 14 causing protrusions 18 and possibly filaments 20 to gently engage the ear canal. Rotation or axial movement of the ear cleaning device when the ear engagement portion 14 in its expanded orientation engages the ear canal and enables ear wax to be entrained by protrusions 18 and possibly by filaments 20. Subsequent withdrawal of the ear cleaning device enables the entrained ear wax to be removed from the ear canal.

It will be appreciated by persons skilled in the art that the present invention is not limited to the claims which follow but also includes combinations and subcombinations of features described hereinabove as well as modifications thereof which would occur to persons skilled in the art upon reading the foregoing description and which are not in the prior art.

1. An ear cleaning device comprising:
a body including a finger engagement portion, a connecting neck portion and an ear engagement portion,
said body being configured such that finger pressure exerted on said finger engagement portion produces expansion of said ear engagement portion.

2. An ear cleaning device according to claim 1 and wherein said ear engagement portion is provided with at least one ear canal engagement enhancing protrusion on an outer surface thereof.

3. An ear cleaning device according to claim 2 and wherein said ear engagement portion is provided with brushlike filaments for ear wax engagement.

4. An ear cleaning device according to claim 3 and wherein said ear engagement portion is inflatable by compression of said finger engagement portion.

5. An ear cleaning device according to claim 3 and wherein said finger engagement portion, connecting neck portion and ear engagement portion are integrally formed of a resilient material.

6. An ear cleaning device according to claim 5 and wherein said connecting portion is less resilient than said finger engagement portion and said ear engagement portion.

7. An ear cleaning device according to claim 2 and wherein said finger engagement portion has a generally bulbous configuration.

8. An ear cleaning device according to claim 2 and wherein said finger engagement portion has a larger interior volume than ear engagement portion.

9. An ear cleaning device according to claim 2 and wherein said finger engagement portion is formed with at least one finger engagement enhancing protrusion on an outer surface thereof.

10. An ear cleaning device according to claim 2 and wherein in use, said ear engagement portion is adapted to be inserted into an ear canal when in a first, generally non-expanded configuration and is adapted to be expanded to gently engage the ear canal.

11. An ear cleaning device according to claim 2 and wherein said ear engagement portion is inflatable by compression of said finger engagement portion.

12. An ear cleaning device according to claim 2 and wherein said finger engagement portion, connecting neck portion and ear engagement portion are integrally formed of a resilient material.

13. An ear cleaning device according to claim 1 and wherein said ear engagement portion is provided with brushlike filaments for ear wax engagement.

14. An ear cleaning device according to claim 1 and wherein said ear engagement portion is inflatable by compression of said finger engagement portion.

15. An ear cleaning device according to claim 1 and wherein said finger engagement portion, connecting neck portion and ear engagement portion are integrally formed of a resilient material.

16. An ear cleaning device according to claim 15 and wherein said connecting portion is less resilient than said finger engagement portion and said ear engagement portion.

17. An ear cleaning device according to claim 1 and wherein said finger engagement portion has a generally bulbous configuration.

18. An ear cleaning device according to claim 1 and wherein said finger engagement portion has a larger interior volume than ear engagement portion.

19. An ear cleaning device according to claim 1 and wherein said finger engagement portion is formed with at least one finger engagement enhancing protrusion on an outer surface thereof.

20. An ear cleaning device according to claim 1 and wherein in use, said ear engagement portion is adapted to be inserted into an ear canal when in a first, generally non-expanded configuration and is adapted to be expanded to gently engage the ear canal.
